(19) United States
(12) Patent Application Publication
(10) Pub. No.: US 2024/0261467 A1
(43) Pub. Date: Aug. 8, 2024

(54) NANOLAMINATE COATING, RELATED COATED ITEMS AND USES, METHOD FOR IMPROVING RESISTANCE OF A SUBSTRATE TO CORROSION IN ESSENTIALLY SALINE ENVIRONMENTS, MEDICAL DEVICE

(71) Applicant: PICOSUN OY, Espoo (FI)

(72) Inventors: Riina RITASALO, Espoo (FI); Tom BLOMBERG, Vantaa (FI)

(21) Appl. No.: 18/561,632

(22) PCT Filed: May 16, 2022
(86) PCT No.: PCT/EP2022/063195

§ 371 Date: Nov. 16, 2023

(30) Foreign Application Priority Data
May 17, 2021 (FI) 20215577

(51) Int. Cl. A61L 27/30; C23C 16/40; C23C 16/455

(52) U.S. Cl. CPC A61L 27/30; C23C 16/402; C23C 16/403; C23C 16/405; C23C 16/45529; A61L 2300/404; A61L 2300/608; A61L 2400/12; A61L 2420/02; A61L 2420/08

(57) ABSTRACT

A corrosion resistant coating for substrates susceptible to corrosion in essentially saline environments, optionally, in vivo environments, is provided. The coating is provided as a nanolaminate structure comprising a plurality of deposition layers formed through a process of chemical deposition in vapour phase, preferably, through Atomic Layer Deposition (ALD) such that the deposition layers having a first composition alternate with the deposition layers having a second composition different from the first composition. A nanolaminate stack produced thereby forms a diffusion barrier that efficiently prevents corrosive species, such as corrosive ionic species, originating from essentially saline environments from contacting the substrate. Related method for improving resistance of a substrate to corrosion in essentially saline media and uses of the nanolaminate coating are further provided.

### FIELD OF THE INVENTION

The present invention generally relates to manufacturing of coated items by chemical deposition methods. In particular, the invention concerns deposition of multilayer laminate coatings onto substrates to render the substrates resistant to corrosion in an essentially saline media.

### BACKGROUND OF THE INVENTION

Chemical deposition methods in vapour phase, such as Chemical Vapor Deposition (CVD) and Atomic Layer Deposition (ALD), are extensively described in the art. ALD technology, generally regarded as a subclass of CVD processes, has proved an efficient tool for manufacturing high-quality conformal coatings on a variety of three-dimensional substrate structures.

ALD is based on alternating self-saturative surface reactions, wherein different reactants (precursors) provided as molecular compounds or elements in a nonreactive (inert) gaseous carrier are sequentially pulsed into a reaction space accommodating a substrate. Deposition of a reactant is followed by purging the substrate by inert gas. Conventional ALD cycle (a deposition cycle) proceeds in two half-reactions (pulse first precursor-purge; pulse second precursor-purge), whereby a layer of material (a deposition layer) is formed in a self-limiting (self-saturating) manner, typically being 0.05-0.2 nm thick. The cycle is repeated as many times as required for obtaining a film with a predetermined thickness. Typical substrate exposure time for each precursor ranges within 0.01-1 seconds. Common precursors include such those that are used to deposit metal oxides, elemental metals, metal nitrides and metal sulfides.

Corrosion protection of structural and electrical components is essential in increasing the lifetime and to reduce failure rates of these components. Corrosion, the gradual degradation of materials by electrochemical attack, became an issue of particular concern in the field of fabrication of so-called bioelectronic devices, in particular, the devices implantable into a patient body.

In the human body, for example, the aqueous medium consists of various anions such as hydroxide, chloride, phosphate, sulphate, and bicarbonate ions, cations (Na⁺, K⁺, Ca²⁺, Mg²⁺ etc.), organic substances of low-molecular-weight species, dissolved oxygen, and the like.

The implantable devices face a severe corrosive environment, generated by virtue of the above mentioned ionic and molecular species, and thus require hermetic encapsulation coatings suitable to withstand diffusion of corrosive elements and deterioration of the device. However, even minor cracks on the coating surfaces may render a biomedical device, such as a bioimplant, exposed to corrosive environments, nonfunctional. In the worst scenario, failure of the biomedical device, such as a cardiac pacemaker or a monitor, for example, may threaten the life of a patient.

Medical/surgical implants typically made of metals and alloys, such as stainless steels, titanium and its alloys, are also often prone to failures due to localized corrosion and replacement of the implant is often associated with a revision surgery. Surgical grade implants (used to fix bones, for example) may release, upon corrosion in human body environments, Fe, Cr and Ni ions, which are powerful allergens.

The basic requirements for successful operation of implantable devices in the body are chemical stability, mechanical behavior and biocompatibility in bodily fluids and tissues. Meeting these requirements is often challenging and, in most instances, solved via a thorough selection of implant materials and/or surface coating and the careful selection of techniques for surface modification (e.g. laser processing, ion implantation). However, compliance with biocompatibility requirements, often set by strict regional standards, may still impose a prominent challenge to manufacturers of implantable bioelectronics solutions. Taken into account that a vast majority of body implantable medical devices are designed for long-term use in the human body, from a few months to several years, biocompatibility of related materials is one of the most important issues to study and develop.

In this regard, an update in the field of fabricating corrosion resistant coatings is still desired, in view of addressing challenges associated with the application of said coatings in manufacturing of biomedical devices, such as implantable bioelectronics solutions.

### SUMMARY OF THE INVENTION

An objective of the present invention is to solve or to at least alleviate each of the problems arising from the limitations and disadvantages of the related art. The objective is achieved by various embodiments of a laminate coating according to what is defined in independent claim 1.

In an embodiment, a laminate coating for substrates susceptible to corrosion in essentially saline environments is provided, the coating comprising a plurality of deposition layers formed through a process of chemical deposition in vapour phase, preferably, through Atomic Layer Deposition (ALD), such that the deposition layers having a first composition alternate with the deposition layers having a second composition different from the first composition, wherein said plurality of deposition layers form a diffusion barrier that prevents corrosive species, such as corrosive ionic species, originating from essentially saline environments from contacting the substrate.

In an embodiment, the individual deposition layers in said laminate coating at least partially block the diffusion of corrosive species therethrough and optionally possess chemical reactivity towards said species. In some configurations, the laminate coating is arranged to hermetically block the diffusion of corrosive species at a surface of the coating.

In an embodiment, the laminate coating comprises at least one ion-conductive or selectively permeable deposition layer enabling selective diffusion of corrosive species originating from essentially saline environments therethrough. The laminate coating further comprises at least one deposition layer reactive to corrosive species originating from essentially saline environments and optionally diffused through the conductive deposition layer(s).

In an embodiment, in said laminate coating the ion-conductive deposition layers alternate with the deposition layers reactive to diffusing corrosive species.

In an embodiment, in said laminate coating, interaction between said reactive deposition layer and the corrosive species originating from the essentially saline environment and optionally diffused through the ion-conductive deposition layer yields formation of at least one additional barrier layer having composition different from that of any one of the deposition layers forming the laminate coating.

In an embodiment, said additional barrier layer is formed at an interface between the ion-conductive deposition layer and the deposition layer reactive to diffusing corrosive species.

In embodiments, the individual deposition layers in said laminate coating are composed of any compound selected from the group consisting of: aluminium oxide(III) (Al₂O₃), titanium(IV) oxide (TiO₂), hafnium(IV) oxide (HfO₂), tantalum(V) oxide (Ta₂O₅), zirconium(IV) oxide (ZrO₂), and silicon dioxide (SiO₂).

In an embodiment, the laminate coating comprises the deposition layers composed of aluminium oxide(III) (Al₂O₃) alternating with the deposition layers of zirconium(IV) oxide (ZrO₂).

In an embodiment, the laminate coating comprises a deposition layer composed of aluminium(III) oxide (Al₂O₃) as a substrate adjacent layer. In embodiments, said Al₂O₃ layer adjoins the substrate and acts as an adhesion layer to mediate or at least to facilitate adhesion of the laminate coating to substrate. In embodiments, the Al₂O₃ layer is deposited directly on the substrate surface.

In another embodiment, the laminate coating comprises the deposition layers composed of hafnium(IV) oxide (HfO₂) alternating with the deposition layers of silicon dioxide (SiO₂).

The laminate coating may have thickness within a range of about 10 nm to about 1000 nm, preferably, within a range of about 50 nm to about 500 nm. In configurations, the laminate coating has thickness within a range of about 10 nm to about 300 nm, preferably, within a range of about 50 nm to about 150 nm.

In an aspect, a laminate coating for substrates susceptible to corrosion in essentially saline environments is provided, the coating comprising a plurality of deposition layers formed through a process of chemical deposition in vapour phase, preferably, through Atomic Layer Deposition (ALD), such that the deposition layers having a first composition alternate with the deposition layers having a second composition different from the first composition, wherein said plurality of deposition layers form a diffusion barrier that prevents corrosive species, such as corrosive ionic species, originating from essentially saline environments from contacting the substrate, and wherein the coating comprises a deposition layer composed of aluminium(III) oxide (Al₂O₃), which adjoins the substrate and acts as an adhesion layer.

In an aspect, a method for improving resistance of a substrate to corrosion in essentially saline environments is provided according to what is defined in independent claim 13.

In a further aspect, use of the laminate coating, according to the embodiments, as a barrier to diffusion of corrosive species, such as corrosive ionic species, towards the substrate in essentially saline environments, optionally in vivo environments, is provided according to what is defined in independent claim 26.

In still further aspect, use of the laminate coating, according to the embodiments, for extending the lifetime of a substrate exposed to essentially saline environments, optionally in vivo environments, is provided according to what is defined in independent claim 27.

Aspects of the invention further concern an item, comprising the laminate coating implemented according to the embodiments, in accordance to what is defined in independent claim 28; and a medical device, such as an implantable medical device, in accordance to what is defined in independent claim 29.

The utility of the present invention arises from a variety of reasons depending on each particular embodiment thereof.

Deposition layers deposited by ALD methods are pinhole-free and fully conformal, therefore, the ALD technology has a high potential in manufacturing of high-quality coatings required for various applications, in particular, medical applications. Due to relatively low temperatures utilized during ALD depositions (100-150° C.), the coatings can be conformally deposited also on sensitive substrate materials. Additionally, since the ALD coatings are generally amorphous (not crystalline), these provide no easy pathway for corrosive species, such as corrosive ionic species, therethrough.

Overall, the invention offers a corrosion resistance (nano)laminate coating for the substrates sensitive to corrosion in essentially saline media, including bodily fluids. A multilayer coating forms an ion diffusion barrier efficiently preventing ionic species that typically cause corrosion in saline media (e.g. Na⁺, K⁺, H⁺; Cl⁻, OH⁻, PO₃³⁻, PO₄³⁻, SO₄²⁻, CO₃²⁻, etc.) from reaching the substrate. Additionally, multilayer coatings reduce formation of cracks and grain boundaries that reducing a number of active diffusion paths through the coating film. Due to the laminate structure realized as a plurality of deposition layers forming a “stack”, the diffusion paths cannot be formed to penetrate the entire layer assembly, therefore, a higher barrier performance can be achieved.

The (nano)laminate coating is particularly suitable for use in medical segment, such as for implantable electronics in cardiology and neurology, to serve as a protective, non-toxic and long-lasting medical coating solution. Substrates coated with the (nano)laminate coatings according to the present disclosure possess resistance to corrosive environments (essentially saline media) even in extreme test conditions (different trials performed during 1 month to 2 months at 87° C. in PBS; and during 100 days at 85° C. in PBS), which is indicative on their potential to be used as coatings for medical devices. The results obtained during the experimental trials have demonstrated that the (nano)laminate coatings disclosed herewith possess equivalent lifetimes of 5 to about 15 years in body fluid environments at 37° C.

Improved reliability in operation and increased lifetimes are of particular importance for implantable medical devices, since replacement of an implant is often associated with surgery; therefore, improving reliability of such devices along with performance characteristics is highly beneficial both for patients and for the healthcare systems.

The laminate coating solutions provide full encapsulation for the substrates even in an absence of auxiliary metal- and/or polymeric coating materials.

The proposed (nano)laminate coatings have proved biocompatible. All coatings have demonstrated low-endotoxin release values less than 2.15 EU/device in accordance with a number of international standards. Moreover, some coatings have demonstrated even lower endotoxicity values within a range of 0.075-0.1 EU/Device.

Additionally, all coatings possess pronounced antimicrobial properties. Based on cytotoxicity, endotoxicity, antimicrobial properties, barrier performance, hemocompatibility, bioburden and other characteristics, the proposed (nano)laminate coatings provide a smart and cost-effective solution for manufacturing coated articles for use in a variety of biotech industries and, in particular, in fabrication of high-performance, optionally nano-scale medical devices compatible with body environment over the decades of use.

In the present disclosure, materials with a layer thickness below 1 micrometer (μm) are referred to as “thin films”.

The expressions “reactive fluid” and “precursor fluids” are indicative in the present disclosure of a fluidic flow comprising at least one chemical compound (a precursor compound), hereafter, a precursor, in an inert carrier.

The expressions “saline media” and “saline environment” are used in the present disclosure to indicate a variety of media that contains dissolved salt compounds. In terms of alkalinity the saline media can be any one of acidic, neutral or alkaline.

In the present disclosure, the term “biocompatibility” is used in its common meaning, viz. defined as an ability of a device or a system of technical nature exposed to biological environment to perform its function without major and clinically adverse manifestations both short and long term. Assessing biocompatibility largely involves analyzing the ability of materials constituting the device/system to interact with biological environment without developing unwanted inflammation or complications in the body.

In the present disclosure, the term “body” (in the context of the expressions “body implantable”, “body environment”, etc.) is used primarily with regard to a human. However, the concept of the present invention is fully applicable to the items, such as medical devices, designed and/or used with a nonhuman mammal or other animal.

The expression “a number of” refers herein to any positive integer starting from one (1), e.g. to one, two, or three; whereas the expression “a plurality of” refers herein to any positive integer starting from two (2), e.g. to two, three, or four.

The terms “first” and “second” are not intended to denote any order, quantity, or importance, but rather are used to merely distinguish one element from another, unless explicitly stated otherwise.

### BRIEF DESCRIPTION OF THE DRAWINGS

FIGS. 1A and 1B schematically illustrate a laminate coating 10, 10A, according to the embodiments, on a substrate 20.

FIGS. 2A and 2B are bright and dark field Scanning Transmission Electron Microscopy (TEM/STEM) images showing cross-sections of the laminate coating 10, according to the embodiment (reference sample and PBS soaked sample, respectively).

FIGS. 3A and 3B are Energy dispersive X-ray spectroscopy (EDS) line profiles for the laminate coating 10, according to the embodiment (reference sample and PBS soaked sample, respectively).

FIG. 4A is a Secondary-ion Mass Spectrometry (SIMS) comparison between sodium (Na⁺) and kalium (K⁺) ion concentration in the laminate coating 10, according to the embodiment (reference sample and PBS soaked sample); while FIG. 4B is a SIMS comparison between the concentrations of chlorine (Cl−) and phosphate (P) ionic species.

FIG. 5 shows bright field TEM images of cross-sections of the laminate coating 10A, according to the embodiment (reference sample and PBS soaked sample).

FIGS. 6A and 6B are EDS mapping images for the laminate coating 10A, according to the embodiment (reference sample and PBS soaked sample, respectively).

FIGS. 7A and 7B are SIMS depth profiles for the laminate coating 10A, according to the embodiment (reference sample and PBS soaked sample, respectively).

### DETAILED DESCRIPTION OF THE EMBODIMENTS

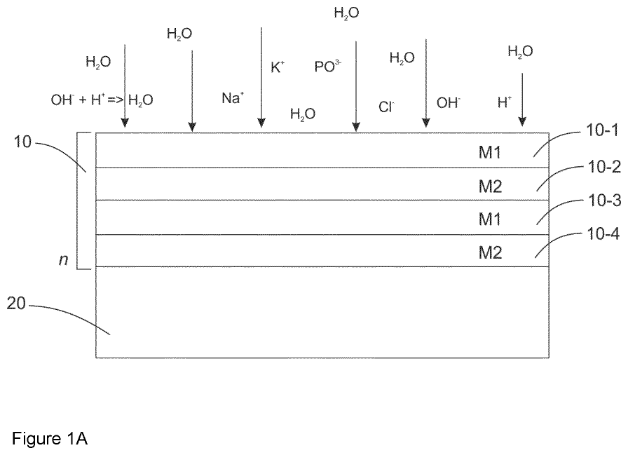

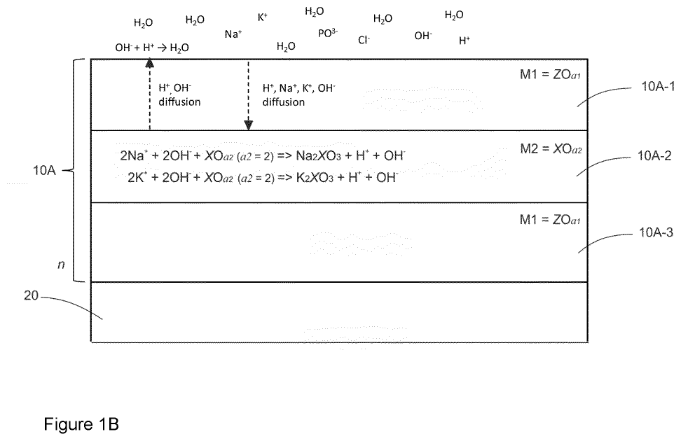

FIGS. 1A and 1B illustrate, at 10 and 10A, respectively, a laminate coating, hereafter, a coating, in accordance with the embodiments.

The coating 10, 10A is advantageously designed for substrates susceptible to corrosion in essentially saline environments, such as for the substrates intended for exposure to corrosive media that contains dissolved salts.

Salts are chemical compounds consisting of an ionic assembly of cations (positively charged ions) and anions (negatively charged ions). In aqueous solutions, salts dissociate with separation of anions and cations. Ionic species are the most common corrosive agents in essentially saline aqueous media.

The examples of essentially saline corrosive media include, but are not limited to commonly used aqueous solutions of salts (buffer solutions, such as phosphate buffered saline (PBS), sodium chloride, etc.), seawater, as well as a variety of in vitro (e.g. cell cultures) and in vivo media. In vivo corrosive media is represented with body fluids and tissue.

The coating 10, 10A comprises a plurality of deposition layers (cf. layers 10-1 to 10-4 on FIG. 1A; and layers 10A-1 to 10A-3 on FIG. 1B) arranged atop each other to form a “stack”. For the sake of clarity, we note that a total number (n) of deposition layers may vary dependent on layer composition, a substrate to be coated and an application field of the latter. By way of example, total number (n) of deposition layers may vary within a range of 2-100. In most instances, n varies within a range of 3-20. A 300 nm stack, for example, can be deposited in 60 deposition cycles, wherein a “sub-stack” of two sublayers (M1, M2) is deposited in each deposition cycle with each material layer having thickness of 2.5 nm. A (nano)laminate structure is thus formed according to a formula: (2.5 nm+2.5 nm)ₙ, wherein n=60. For a skilled person it is clear that dependent on reaction conditions one may deposit a plurality of 1-3 nm sublayers, for example, thus regulating the total stack thickness/depth of the nanolaminate stack.

In the coating 10, 10A, a stack formed with a plurality of deposition layers has thickness within a range of about 10 nm to about 1000 nm; therefore, the laminate structure presented herewith is also referred to as “nanolaminate”. With regard to the coating 10, 10A, the expressions “a structure” (nanolaminate structure) and “a stack” (nanolaminate stack) are used in the present disclosure interchangeably. Nanolaminate stacks having thicknesses within a range of 10 nm to 1000 nm, preferably, within a range of 50 nm to 500 nm, still preferably, within a range of 100 to 300 nm, can be produced. Most typical and, in some instances, preferred ranges for the deposited nanolaminate structures 10, 10A include 50-300 nm, namely, 50 nm, 100 nm, 150 nm, 200 nm, 250 nm, and 300 nm. Still, nanolaminate structures having thicknesses exceeding 300 nm (up to about 1000 nm) can be produced in a predetermined number (n) of deposition cycles.

As shown on FIGS. 1A and 1B, a topmost laminate layer 10-1, 10A-1 is in direct contact with the surrounding media, while a lowermost layer 10-4, 10A-3 is deposited over a surface of a substrate 20, optionally, over an adhesive layer (not shown). Nevertheless, in majority of laminate coatings according to the present disclosure, it is the lowermost layer that provides adhesion to substrates. A number of ALD films have been studied for their capacity to adhere to the substrate.

Deposition layers are formed on the substrate through a process of chemical deposition in vapour phase, preferably, through Atomic Layer Deposition (ALD).

The basics of an ALD growth mechanism are known to a skilled person. ALD is a special chemical deposition method based on the sequential introduction of at least two reactive precursor species to at least one substrate. It is to be understood, however, that one of these reactive precursors can be substituted by energy when using, for example, photon-enhanced ALD or plasma-enhanced ALD, for example PEALD, leading to single precursor ALD processes. For example, deposition of a pure element, such as metal, requires only one precursor. Binary compounds, such as oxides can be created with one precursor chemical when the precursor chemical contains both of the elements of the binary material to be deposited. Thin films grown by ALD are dense, pinhole free and have uniform thickness.

In ALD, the at least one substrate is typically exposed to temporally separated precursor pulses in a reaction vessel to deposit material on the substrate surfaces by sequential self-saturating surface reactions. In the context of present disclosure, the term ALD comprises all applicable ALD based techniques and any equivalent or closely related technologies, such as, for example the following ALD sub-types: plasma-assisted ALD, PEALD (Plasma Enhanced Atomic Layer Deposition) and photon-enhanced Atomic Layer Deposition (known also as photo-ALD or flash enhanced ALD).

A deposition setup may be the one based on an ALD installation described in the U.S. Pat. No. 8,211,235 (Lindfors), for example, or on the installation trademarked as Picosun R-200 Advanced ALD system available from Picosun Oy, Finland. Nevertheless, the features underlying a concept of the present invention can be incorporated into any other chemical deposition reactor embodied as an ALD, PEALD, Molecular Layer Deposition (MLD), or a Chemical Vapour Deposition (CVD) device, for example.

An exemplary ALD reactor comprises the reaction chamber that establishes the reaction space (deposition space), in which the production of nanolaminate coatings described herewith takes place. The reactor further comprises a number of appliances configured to mediate fluidic flow (inert fluids and reactive fluids containing precursor compounds P1, P2) into the reaction chamber. These appliances are provided as a number of intake lines/feedline and associated switching and/or regulating devices, such as valves, for example.

A basic ALD deposition cycle consists of four sequential steps: pulse A, purge A, pulse B and purge B. Reactive fluid entering the reaction chamber during pulses A and B is preferably a gaseous substance comprising a predetermined precursor chemical (P1, P2) carried by an inert carrier (gas). Delivery of the precursor chemicals into the reaction space and film growth on the substrate is/are regulated by means of the abovesaid regulating appliances, such as e.g. three-way ALD valves, mass-flow controllers or any other device suitable for this purpose.

The above described deposition cycle can be repeated until the deposition sequence has produced a thin film or coating of desired thickness. Deposition cycles can also be either simpler or more complex. For example, the cycles can include three or more reactant vapor pulses separated by purging steps, or certain purge steps can be omitted. On the other hand, photo-enhanced ALD has a variety of options, such as only one active precursor, with various options for purging. All these deposition cycles form a timed deposition sequence that is controlled by a logic unit or a microprocessor.

The ALD process utilized to produce (nano)laminates 10, 10A employs deposition temperatures within a range of about 80-250° C. Deposition within a range of 100-150° C. enables or facilitates coating of heat-sensitive substrates, such as those containing polymers and/or electric circuits.

With reference to FIGS. 1A, 1B, an individual deposition layer (10-1 to 10-n; 10A-1 to 10A-n) may be formed in one deposition cycle (with a sequence of P1-purge P2-purge), as a monolayer. In most instances, a number of sequential deposition cycles can be employed to produce a deposition layer (e.g. 10-1 or 10A-1) of desired thickness.

The laminate coating 10, 10A is formed with a plurality of deposition layers, in which the layers (e.g. 10-1, 10-3; 10A-1, 10A-3) having a first composition alternate with the deposition layers (e.g. 10-2, 10-4; 10A-2, respectively) having a second composition different from the first composition.

In the coatings 10, 10A shown on FIGS. 1A and 1B the layers deposited with a first compound or a group of compounds (M1 standing for “Material 1”) alternate with the layers deposited with a second compound or a group of compounds (M2 standing for “Material 2”), non-identical to the first one(s).

To produce the nanolaminate structures 10, 10A, the deposition layers were produced from a selection of (metal) oxide species including aluminium(III) oxide (Al₂O₃), titanium(IV) oxide (TiO₂), hafnium(IV) oxide (HfO₂), tantalum(V) oxide (Ta₂O₅), zirconium(IV) oxide (ZrO₂), niobium(V) oxide (Nb₂O₅), and silicon(IV) dioxide (SiO₂). Metal nitride compounds, such as titanium nitride, aluminium nitride or silicon nitride, for example, can also be utilized. Utilization of any other appropriate compound is not excluded.

In the laminate structure 10, 10A, the plurality of deposition layers forms a barrier that prevents corrosive species originating from essentially saline environments from penetrating deep into the structure and from contacting the substrate. In essentially saline media, the corrosive species are typically ions; however, diffusion of e.g. low-molecular compounds into the structure can also be prevented.

In some configurations, by virtue of combination of deposition layers having different chemical compositions, the coating 10, 10A acts as a diffusion barrier that efficiently protects the substrate from corrosive media. A plurality of deposition layers in the coating 10, 10A can thus be configured to form an ion diffusion barrier.

In terms of (chemical) composition of the individual deposition layers, the coating is designed such as to at least partially block the diffusion of corrosive species, such a corrosive ionic species, through said layers and optionally possess chemical reactivity towards the diffusing ionic species.

In some configurations, the coating is designed to fully block the diffusion of corrosive species at a surface of the nanolaminate, viz. at a surface boundary between the upmost nanolaminate layer and the surrounding media).

Ionic species normally occurring in saline environments have different sizes and different (molar) ionic conductivity. The smaller the bare ion size, the greater its expected diffusion rate through ion-conductive (also referred to as selectively permeable or ion-selective) material layers. Thus, ionic species having smaller size (e.g. Li⁺, Na⁺, K⁺, NH₄+, H⁺, OH⁻, Cl⁻, etc.) penetrate through the ion-conductive materials faster than larger ionic species (e.g. PO₄³⁻, PO₃³⁻, NO₃⁻, ClO₄⁻, SO₄²⁻, etc.). Also the charge of ionic species effects the diffusion rate through the materials (positive- and negative ion conductors). Additionally, presence of an electric field at a vicinity of material interfaces can modify the ionic conductivity of materials, in particular, of thin films, as it may cause a drift force for the ions, depending on the sign of the charge at the interface.

The present disclosure thus presents multilayer structures for providing fully hermetic coatings that efficiently block diffusion of corrosive species, such as corrosive ionic species, at the laminate surface; and coatings that form an ionic diffusion barrier through chemical interactions occurring inside the laminate structure.

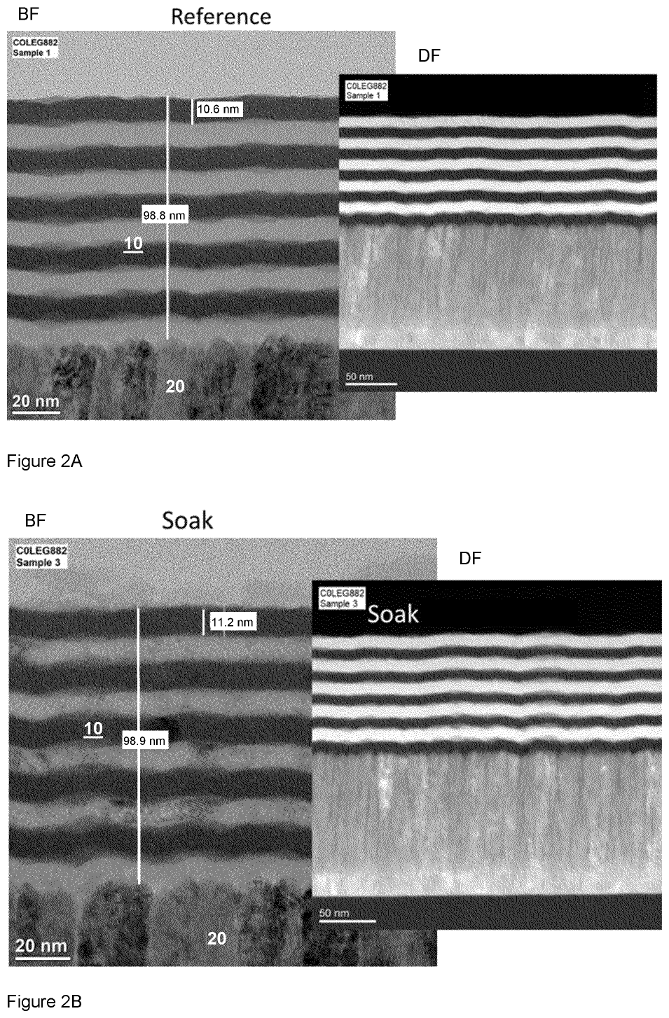

FIGS. 2-4 illustrate a concept of a so-called hermetic laminate coating designed to block the diffusion of corrosive (e.g. ionic) species at a surface of the laminate. The coating is implemented according to a configuration shown in FIG. 1A.

By way of example, the coating 10 was implemented on a substrate as a layered structure comprising the alternating deposition layers composed of aluminium oxide(III) (Al₂O₃) and zirconium(IV) oxide (ZrO₂).

Laminate nanolayers were deposited through ALD using tetramethylammonium (TMA) and H₂O precursors to deposit Al₂O₃ layers and Tetrakis(ethylmethyl-amino)zirconium (TEMAZr) and H₂O precursors to deposit ZrO₂ layers.

FIGS. 2A and 2B are bright field (BF) and dark field (DF) TEM/STEM images of the coating 10, according to the embodiment, on a substrate 20 before (FIG. 2A) and after (FIG. 2B) a 2-month long exposure to a PBS solution (at 87° C.). The coating 10 is realized as a Al₂O₃—ZrO₂ nanolaminate coating. In BF images the bright layer is Al₂O₃ and the dark layer is ZrO₂. In DF images these are opposite. The layers were ALD deposited at 150° C. on Si/SiO₂ chips with TiN conducting lines (substrate size approximately 3 cm×1 cm).

No difference has been noticed between the soaked sample and the reference, which is indicative of the coating 10 being fully hermetic.

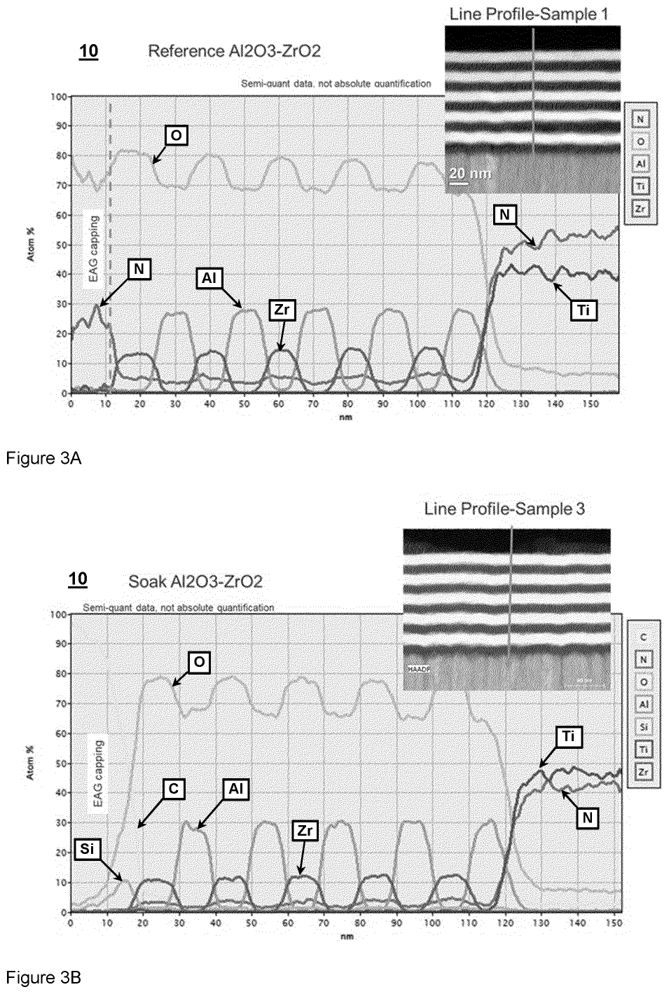

FIGS. 3A and 3B show EDS line profile for the reference sample and the soaked sample accordingly. The laminate coating 10, the substrate and the soaking conditions were the same as described with reference to FIGS. 2A and 2B. From the EDS profiles one may observe that no differences occur, in the reference- and test samples, underneath the topmost layer.

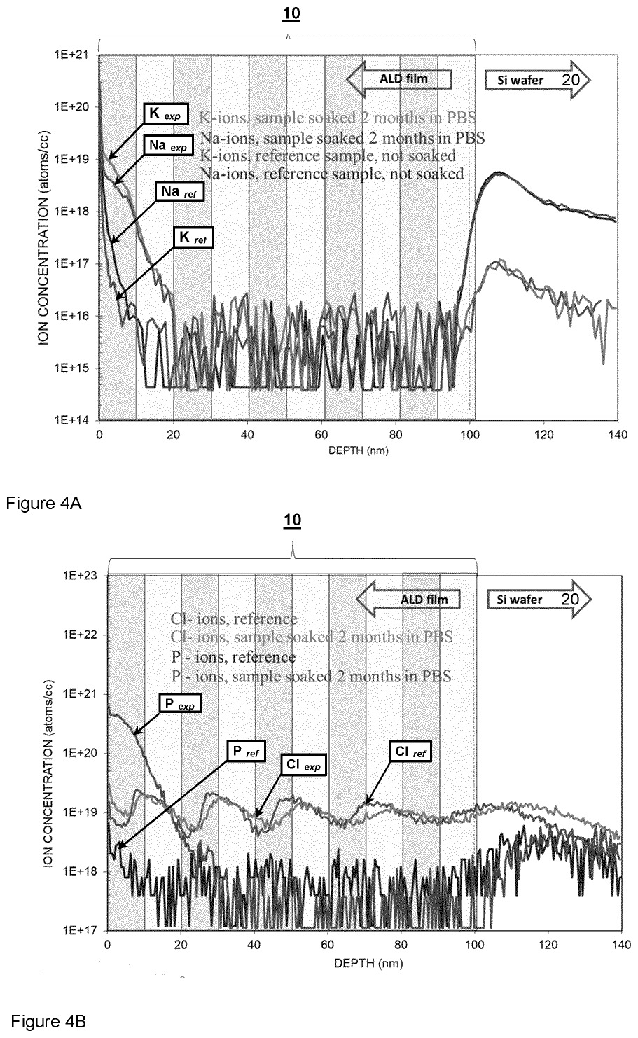

FIGS. 4A and 4B are SIMS graphs showing a comparison between cation− (Na⁺, K⁺ species) and anion− (Cl⁻, PO₄³⁻ species) concentrations, respectively, in the reference sample and in the test sample (soaked sample). The laminate coating 10, the substrate and the soaking conditions were the same as described above (FIGS. 2 and 3). Na and K ionic contents on the sample surface were slightly higher for the exposed sample; however, at a depth of about 20-30 nm (third deposition layer from the top), no differences between the exposed sample and the reference could be observed.

The results of FIGS. 4A and 4B confirm that the nanolaminate film 10 (about 100 nm), comprising a plurality of alternating Al₂O₃—ZrO₂ layers, efficiently blocks diffusion of ionic species most commonly occurring in saline environments (e.g. Na⁺, K⁺, Cl⁻, PO₄³⁻) thus preventing said ionic species from contacting the substrate. FIG. 4A also shows that a concentration of Na⁺ and K⁺ ionic species inside the ALD film is even lower that the concentration of said species in an underlying substrate (Si wafer).

It should be noted that the 2-months tests conducted at 87° C. in PBS (so called accelerated exposure tests) correlate with about 5.3 years exposure in bodily fluids environments at 37° C.

Moreover, accelerated exposure tests with on-line resistance measurements conducted for five samples (Si/SiO₂/Al chips) coated with Al₂O₃—ZrO₂ nanolaminate (ALD-deposited at 125° C.) for a period of 100 days at 85° C. in PBS have demonstrated an absence of corrosion in all tested samples at the end of the trial (not shown). These exposure conditions correlate with more than an 8-year period in a body fluids environment at 37° ° C.

Formation of a hermetic nanolaminate coating 10 can also be achieved with a nanolaminate coating 10, including, but not limited to combinations of Al₂O₃ and/or ZrO₂ with any one of TiO₂, HfO₂, Ta₂O₅ and SiO₂.

Amongst combinations cited above, also Al₂O₃—HfO₂ nanolaminates and Al₂O₃—Ta₂O₅ nanolaminates have proved highly resistant to corrosion when exposed to corrosive saline environments. PBS soak tests (4-8 weeks in PBS at 87° C.) were conducted on Si/SiO₂ chips with TiN conducting lines in similar manner as described with regard to FIGS. 2A and 2B. Similarly to Al₂O₃—ZrO₂ nanolaminates shown on FIGS. 2A and 2B, Al₂O₃—HfO₂ and Al₂O₃—Ta₂O₅ laminate coatings included alternating five layers of aluminium oxide and five layers of hafnium oxide or tantalum oxide, respectively (i.e. ten layers in total). In all coatings (Al₂O₃—ZrO₂, Al₂O₃—HfO₂ and Al₂O₃—Ta₂O₅) alumina layer was the lowermost layer that formed the interface with the substrate. Thickness of each ALD layer was about 10 nm and the total thickness of the nanolaminate coating was about 100 nm, resulting from a number of sequential deposition cycles.

TEM analysis on laminate cross-sections has revealed that for example Al₂O₃—HfO₂ nanolaminate has not changed after corrosive treatment (not shown). Thickness of the 5*(10+10) layer laminate was measured as 106.7 nm before the treatment and 106.9 nm after the treatment, and thickness of a topmost hafnia layer was 11.7 nm before- and 11.9 nm after the treatment. The laminate could also be deposited as a 10*(5+5) layer. The coatings have not demonstrated any notable surface defects, which supports the view that different laminate coatings, according to the invention, are fully hermetic.

It was further observed that the alumina (Al₂O₃) layer provided in the nanolaminate coating as a bottom-most layer deposited directly over the substrate facilitates adhesion of the laminate stack on the substrate.

Overall, it has been demonstrated that the lowermost alumina layer provides better adhesion to the substrate than any other tested metal oxide layer. Having alumina as a substrate interface layer, neither delamination nor visible defects was observed in the ALD coating. Hence, according to some embodiments, the nanolaminate coating comprises the aluminium(III) oxide (Al₂O₃) layer deposited on the substrate to adjoin the substrate and to function as an adhesion layer.

Same soak tests (4-6 weeks in PBS at 87° C.) were conducted on samples ALD-coated with individual metal oxides (Al₂O₃, HfO₂ and Ta₂O₅; total thickness of the coating about 100 nm). In these tests, samples coated with pure Al₂O₃ failed in two days (dissolved in PBS); while samples coated with pure Ta₂O₅ and pure HfO₂ demonstrated notable surface defects. This may be due to the observation that ALD coatings deposited with individual metal oxides, such as for example 100 nm HfOₓ films, demonstrate partially crystalline behavior, which increases disorderliness of deposited films and facilitates penetration of ions into/through the film.

However, with formation of the layered (nano)laminate coating structure according to the present disclosure, crystal growth can be prevented completely. Furthermore, the boundaries between the different deposition layers prevent easy access of ion penetration through the entire ALD stack. In addition, it appears that Al₂O₃ plays an integral role in the structure and functionality of the stack.

Additionally, series of tests made for 100 nm nanolaminates: Al₂O₃—ZrO₂, Al₂O₃—HfO₂ and Al₂O₃—Ta₂O₅ coated onto LCP (liquid crystal polymer) substrates using ALD at 125° C. and 150° C. demonstrated that these coatings decrease diffusion of moisture therethrough by up to two orders of magnitude. Results were confirmed with moisture (water) vapor transmission rate (MVTR) measurements. For example, for Al₂O₃—HfO₂ laminate, moisture vapor permeation (diffusion coefficient) decreased from 0.3 um²/s for a bare LCP sheet to 0.003 um²/s.

FIGS. 1B, 5-7 illustrate a concept of a laminate coating (10A), in which formation of the ion diffusion barrier involves chemical interactions between the deposition layers and diffusing ionic species.

The laminate coating 10A (FIG. 1B) comprises at least one ion-conductive (ion-selective) deposition layer that enables selective diffusion of corrosive species therethrough. The laminate coating 10A further comprises at least one deposition layer reactive to corrosive species originating from essentially saline environments and optionally diffused through the ion-conductive selectively permeable deposition layer(s). Said reactive layer may further be rendered with selective reactivity towards diffusing corrosive species.

In an event the corrosive species are ionic species, the selectively permeable layer is referred to as an ion-conductive layer; and the reactive layer is referred to as an ion-reactive layer. Additionally or alternatively, the layers can be designed for being selectively ion-conductive/reactive for molecular or elemental corrosive species occurring in the environment surrounding the coated substrate.

In a described embodiment, the ion-conductive deposition layers are disposed adjacent to the ion reactive layers. In the (nano)laminate stack, these layers alternate.

Configuration illustrated in FIG. 1B describes diffusion of corrosive ionic species into the laminate 10A. In the multilayer structure, an ion-reactive layer (10A-2) is arranged underneath a topmost ion-conductive layer (10A-1). The ion-conductive- and ion-reactive layers alternate in a manner that the reactive layers are deposited between the ion-conductive layers. In present example, a layer (10A-3) underneath the reactive layer 10A-2 is again made ion-conductive. The number of layers (n) vary dependent on particular coating composition, substrate, field of application, etc.

In some instances, the laminate coating 10A may comprise the reactive layer as a topmost layer. In such an event, the corrosive species diffuse from the essentially liquid environment directly into the reactive layer so that diffusion occurs at an interface between the laminate coating (topped with an ion-reactive layer) and the media surrounding the coated substrate.

Interaction between the reactive deposition layer(s) and the corrosive species, such as corrosive ionic species, originating from essentially saline media and optionally diffused through the ion-conductive deposition layer(s) yields formation of at least one additional barrier layer having composition different from that of any one of the deposition layers forming the laminate coating. The interaction may be rendered selective, whereupon the reactive layers will interact only with certain corrosive species. Dependent on a nanolaminate configuration, the additional barrier layer can be formed at an interface between the conductive layer and the deposition layer reactive to diffusing corrosive species. Additionally or alternatively, the additional barrier layer may be formed at a boundary between the reactive deposition layer and a medium surrounding the coated substrate (in an event the reactive layer is deposited atop of the entire laminate structure).

Selective ion-conductivity and optionally selective ion-reactivity towards the corrosive species, such as corrosive ionic species, is achieved by virtue of chemical composition of the deposition layers, i.e. by virtue of compounds or groups of compounds constituting said layers. As mentioned above, the alternating deposition layers are made of distinct materials (M1, M2). In configuration of FIG. 1B, the ion-conductive layer 10A-1 (M1) is composed of a substance ZOₐ₁ and the ion-reactive layer 10A-2 (M2) is composed of a substance XOₐ₂, wherein any one of X and Z are selected from metal species and semiconductor species.

In configuration of FIG. 1B, the corrosive species (ionic species H⁺, Na⁺, K⁺, OH⁻) diffusing from the environment into the ion-conductive layer 10A-1 (M1=ZOₐ₁) reach the adjacent ion-reactive layer 10A-2 (M2=XOₐ₂) and interact with the compound (XOₐ₂) forming said reactive layer 10A-2. Interaction between the reactive deposition layer (the compounds or groups of compounds forming said layer) and the corrosive species selectively diffused through the conductive layer (10A-1) yields formation, inside the laminate coating, of new compound species different from those constituting any one of the ion-conductive- and reactive layers 10A-1, 10A-2. An additional barrier layer having composition different from that of any one of the deposition layers forming the laminate coating is thus formed inside the nanolaminate, at an interface between the reactive layer and the conductive layer (i.e. “sandwiched” between the deposition layers 10A-1, 10A-2).

The additional barrier layer improves ion diffusion barrier efficiency of the laminate coating 10A exposed to essentially saline environments.

In the example of FIG. 1B, the additional barrier layer is sodium- or potassium salt formed according to the following mechanism:

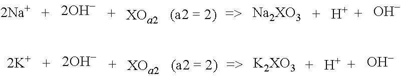

Coefficient a2 varies depending on the oxidation state of the species X forming the compound XOₐ₂. The same stands for the coefficient a1 in the compound ZOₐ₁.

It is assumed that a person skilled in the art shall experience no difficulties in figuring out the interaction mechanisms involving different ionic species or even molecular species, for example, and design reactive deposition layers appropriately.

In exemplary configuration, the coating 10A was implemented on a substrate as a layered structure comprising the alternating deposition layers composed of hafnium(IV) oxide (HfO₂) and silicon dioxide (SiO₂). In such an event, the ion-conductive layers (10A-1, 10A-3) made of Material 1 were composed of HfO₂ (ZOₐ₁=HfO₂); while the ion-reactive layers (10A-2) made of Material 2 were composed of SiO₂ (XOₐ₂═SiO₂).

Formation of an additional barrier layer occurs according to the following scenario:

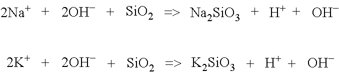

In the presented example, the additional barrier layer consists of a silicate salt. The additional barrier layer prevents, fully or partly, diffusion of corrosive species deeper into the laminate. In some instances, corrosive species, such as corrosive ionic species, may penetrate deeper into the laminate structure and interact with the reactive deposition layers located deeper in the laminate.

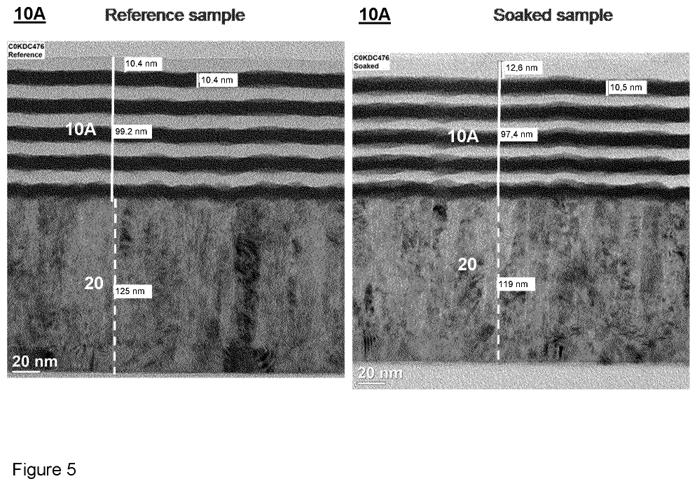

FIG. 5 shows bright field TEM images of the coating 10A, according to the embodiment, on a substrate 20 before (left image) and after (right image) a 1-month exposure to a PBS solution (at 87° C.). The coating 10A is realized as a HfO₂—SiO₂ nanolaminate coating. The layers were ALD deposited on a Si/SiO₂/TiN substrate. The dark layers are ion-conductive layers (HfO₂) and the bright layer are ion-reactive layer (SiO₂). From the experimental image on the right is may be observed that the uppermost deposition layer (ion-reactive layer, SiO₂) has generally increased in its depth/thickness (12.6 nm as compared to 10.4 nm in the reference sample) as a result of interaction of silicon oxide with diffusing ionic species.

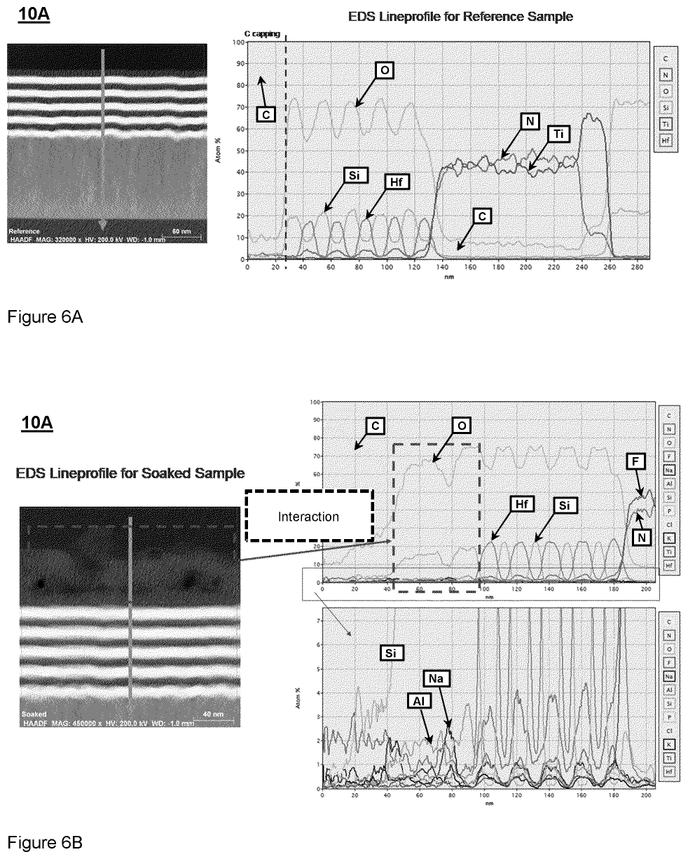

FIGS. 6A and 6B are EDS mapping images for the HfO₂—SiO₂ nanolaminate coating, described with reference to FIG. 5. The topmost layer was SiO₂. From the experimental sample (FIG. 6B) it can be observed that said topmost SiO₂ layer reacted with ionic species originating from essentially saline environment (hereby, PBS buffer solution) and became swollen (see dashed box).

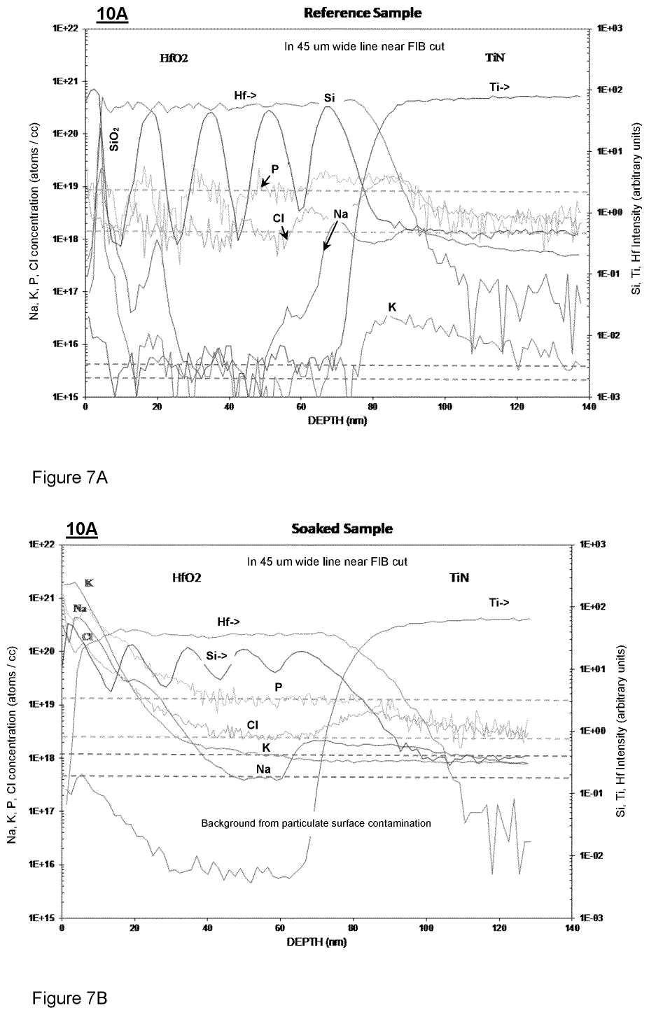

FIGS. 7A and 7B are SIMS depth profiles showing a relative content of ionic species (Na⁺, K⁺, Cl⁻, and PO₄³⁻), in the HfO₂—SiO₂ nanolaminate coating, described with reference to FIGS. 5 and 6, before and after exposure to PBS solution. From FIG. 7B it can be observed that relative content of sodium and potassium ions deep in the nanolaminate has increased substantially, whereas the relative content of chlorine and phosphate ions has increased only slightly or not increased at all.

The results shown on FIGS. 5-7 are thus indicative of formation of a new compound layer inside the laminate coating.

Other examples for formation of an internal barrier layer within the nanolaminate coating 10A include the coatings composed of a plurality of deposition layers with alternating ZrO₂ and SiO₂.

Table 1 below provides a non-exclusive overview of anticorrosive nanolaminate coatings 10, 10A and their ALD deposition temperatures.

TABLE 1
ALD nanolaminate coatings and their
exemplary deposition temperatures.

| Nanolaminate coating | Deposition |
|---|---|
| 10, 10A, 50-300 nm, in | temperature, |
| some examples, 50-150 nm | range (° C.) |
| Al₂O₃—HfO₂ | 85-200/250 |
| Al₂O₃—ZrO₂ | |
| Al₂O₃—SiO₂ | |
| Al₂O₃—Ta₂O₅ | |
| HfO₂—SiO₂ | |
| ZrO₂—SiO₂ | |
| Al₂O₃—TiO₂ | 85-200 |

The examples described hereinabove concerned the (nano)laminate coatings 10, 10A, wherein a deposition layer composed of a certain compound or a group of compounds was duplicated every second layer in a laminate stack. Stacks formed with deposition (sub)layers (M1-M2)n were thus produced. The deposition techniques, such as ALD, employed herewith allow for constructing more complex laminate solutions including three-, four or more different material (M) sublayers in a “sub-stack”, which can be repeated n times as required.

A number of nanolaminates was studied for their capacity to provide electrical barrier/insulation capability closely related to electrical encapsulation, which is particularly important in implantable bioelectric devices (so called smart implants). Thus, silicon substrates coated with Al₂O₃—ZrO₂, Al₂O₃—HfO₂ and Al₂O₃—Ta₂O₅ nanolaminates (ALD deposition 150° C., water process; 5*(10+10)-layer coatings; 100 nm) were tested for their leakage current density at an applied electric field of 6 MV/cm. These tests, indicated that the chosen laminates demonstrated almost zero leakage (in the range of 10-8), thus proving their potential for use in biomedical devices.

In another aspect, a method for improving resistance of a substrate to corrosion in essentially saline environments is provided. In the method, a suitable substrate 20 is deposited with a laminate coating 10, 10A, according to the embodiments described hereinabove.

The coatings are preferably formed through a process of chemical deposition in vapour phase, preferably, through Atomic Layer Deposition (ALD). Overall, a plurality of deposition layers is formed such, that the deposition layers having a first composition alternate with the deposition layers having a second composition different from the first composition. By virtue of depositing said plurality of nanolaminate layers (10-1 to 10-n; 10A-1 to 10A-n), a diffusion barrier is formed that prevents corrosive species, such as corrosive ionic species originating from essentially saline environments from contacting the substrate.

In embodiments, the method comprises depositing a plurality of deposition layers, which at least partially block the diffusion of corrosive species therethrough and optionally possess chemical reactivity towards said species.

In embodiments, formation of the diffusion barrier involves chemical interactions between the deposition layers and diffusing corrosive species.

Thus, in order to produce certain nanolaminate configurations, the method comprises formation of the coating with at least one ion-conductive deposition layer that enables selective diffusion of corrosive species, such as corrosive ionic species, originating from essentially saline environments therethrough and with at least one deposition layer reactive to said corrosive species originating from essentially saline environments and optionally diffused through the conductive deposition layer(s). The conductive layers alternate with the deposition layers reactive to diffusing corrosive species. These layers are formed adjacent to one another in the nanolaminate stack.

In some embodiments, the method further comprises a step of exposing the substrate deposited with the laminate coating to an essentially saline environment. The substrate can be thus immersed into a liquid saline solution (e.g. PBS, NaCl; seawater). The essentially saline environment can be also provided as an in vitro cell culture or the like.

In some particular embodiments, the essentially saline environment is an in vivo environment, such as body fluid or tissue.

In some embodiments, the method further includes formation of an additional barrier layer, when the substrate deposited with the (nano)laminate coating is exposed to said essentially saline environment. In accordance to what has been described above, the additional barrier layer has a composition different from that of any one of the deposition layers forming the laminate coating.

The additional barrier layer may be formed at a boundary between the ion-conductive deposition layer and the deposition layer reactive to diffusing corrosive species. Additionally or alternatively, the additional layers may be formed essentially at a boundary between the media surrounding the coated substrate and the deposition layer of the coating (selectively) reactive to the diffusing corrosive species.

In a further aspect, use of the laminate coating implemented according to the embodiments is provided as a barrier to diffusion of corrosive species, such as corrosive ionic species towards the substrate in essentially saline environments, optionally in vivo environments. In still a further aspect, use of the laminate coating implemented according to the embodiments is provided for extending the lifetime of a substrate exposed to essentially saline environments, optionally in vivo environments.

A number of experimental trials has been conducted to assess resistance of the (nano)laminate coatings 10, 10A to corrosion in essentially saline media. It has been established that the coatings 10, 10A deposited through the ALD process can be used as corrosion resistance layers on medical devices, in particular, on implantable medical devices, including implantable bioelectric devices and stimulating devices, during a prolonged use in vivo.

The coatings 10, 10A can be provided to coat a part of the substrate or the entire substrate. In a latter case, the coating acts as a sealing to encapsulate the entire substrate (e.g. a medical device) and to prevent the device failure by protecting it from the corrosive environment in bodily fluids, for example. At the same time, the coatings 10, 10A prevent leakages of metal ions originating from implantable materials, for example, into the body.

During the accelerated exposure tests (2 months at 87° C. in PBS), the ALD-deposited (nano)laminates consisting of repeating layers of metal oxides (Al₂O₃ and ZrO₂; SiO₂ and HfO₂) have efficiently prevented degradation of the coating itself and simultaneously have prevented the ionic species from penetrating through the deposition layers and from reaching the substrate. Mentioned accelerated exposure tests correlate well with about 5.3 years exposures in bodily fluids environments at 37° C.

To assess biocompatibility of the nanolaminate coatings 10, 10A, experimental trials were conducted employing the following compounds: Al₂O₃, TiO₂ (deposited with different precursors), HfO₂, Ta₂O₅, SiO₂ and ZrO₂. Each of these materials was ALD deposited on silica wafer substrates at 125° ° C. using the R-200 Advance ALD system (Picosun) in clean rooms ISO 6-7 to produce coating films having thickness of approximately 100 nm. These coatings films were analyzed as described further below.

Endotoxicity trials were conducted with the above indicated compounds. The coating films (about 100 nm) were deposited to encapsulate a 100 mm (diameter) silica water (total surface area about 157 cm²). Each sample compound was tested for endotoxicity in replicates of three.

Endotoxicity was assessed using a Limulus Amebocyte Lysate (LAL) test with kinetic turbidimetric method and in accordance with the regulatory documents ANSI/AAMI ST72:2019, USP <161>, USP <85>, EP 2.6.14 and JP 4.01. All studied compounds have passed the endotoxin tests by demonstrating the endotoxin release values (K) within the (allowable) limits set for finished medical devices that will not contact cerebrospinal fluid (K=20 Endotoxin Units (EU)/device) and those set for medical devices that may come into contact with cerebrospinal fluid (K=2.15 EU/device) (cf. USP Chapter <161>). All tested compounds demonstrated average K values of <0.5 EU/device (<0.005 EU/ml with extraction volume of about 100 mL/device).

In addition, follow-up studies for SiO₂ and HfO₂ (with smaller-sized substrates) have confirmed that these compounds fulfill endotoxicity requirements also for intraocular devices. Tested compounds demonstrated K values within a range of 0.075-0.100 EU/Device (<0.005 EU/ml with extraction volume of about 15 ml/device). For intraocular ophthalmic devices, the endotoxin release value limit is no more than 0.2 EU/device; the K values observed for SiO₂ and HfO₂ are clearly lower than the required threshold.

Hence, the nanolaminate coatings according to the present disclosure represent low endotoxin release materials suitable for use with different types of medical devices, including implantable medical devices.

Moreover, it has been established that the coatings 10, 10A have pronounced antimicrobial activity against both gram-negative and gram-positive bacteria. Samples ALD-deposited with the same compounds as used for endotoxicity tests were studied according to the ISO 22196:2011 (Measurement of antibacterial activity on plastics and other non-porous surfaces). Two bacterial strains were used to test the surface antibacterial properties of the ALD-deposited films. Gram-positive bacterial strain was Staphylococcus aureus (S. aureus; ATCC 6538) and gram-negative strain was Escherichia coli (E. coli; ATCC 8739). A non-coated glass plate was used as a reference.

Reference (control) and test surfaces (50 mm×50 mm) were inoculated with microorganisms and the inoculum was covered with a thin sterile film (40 mm×40 mm). Microbial concentrations were determined at “time zero” by elution followed by dilution and plating to agar. Inoculated and covered surfaces were allowed to incubate undisturbed in a humid environment for 24 h±1 h at 36° C.±1° C. After incubation the microbial concentrations were determined and the reduction of microorganisms relative to the control surface was calculated. The determinations were made in double replicate.

According to the results, the Al₂O₃ coating demonstrated reduction in the amount of both S. aureus and E. coli after 24 h contact time, with reduction percentages of 36.00% and 38.03%, respectively.

Overall, Al₂O₃ and SiO₂ demonstrated reduction in the amount of viable microorganisms after the 24 h contact time when tested with S. aureus. The reduction was 36.00% and 37.14%, respectively, when compared to the control sample. With E. coli, Al₂O₃, TiO₂ (1^{st} sample), TiO₂ (2^{nd} sample deposited with different precursors), Ta₂O₅, ZrO₂ and HfO₂ demonstrated the reduction values of: 38.03%, 20.77%, 54.23%, 33.80%, 47.54% and 36.97%, respectively (24 h contact time).

Same compounds as above, provided as ALD films on a silica wafer matrix (100 mm diameter), were additionally tested for total bioburden and for cytotoxicity.

Total bioburden analysis concerned determining the population of microorganisms on the products according to EN ISO 11737-1:2018 standard (Sterilization of health care products—Microbiological methods—Part 1: Determination of a population of microorganisms on products in a DIN EN 150/IEC 17025:2005 accredited laboratory). The results demonstrated that no aerobic bacteria, anaerobic bacteria, yeasts, molds or spores were detected in the products tested under the tested conditions.

Cytotoxicity tests involved, in addition to the compounds cited above, also niobium(V) oxide (Nb₂O₅), titanium nitride (TIN) and aluminium nitride (AlN) films. Cell viability was assessed in-vitro according to the ISO 10993-5 standard, designed to determine the biological response of mammalian cells in vitro using appropriate biological parameters. Cultured cells (BJ fibroblast cell line: human diploid foreskin fibroblasts) were incubated in contact with a tested sample for 72 h in a medium containing 10% FBS (Fetal Bovine Serum) at 37° C. The results confirmed that all test compounds are non-cytotoxic.

Same compounds as those tested for endotoxicity, provided as ALD films on a silica wafer matrix, were further analyzed for direct and indirect hemolysis according to the standard ISO 10993-4:2017. Reference sample was a non-coated Si-wafer. Hemolysis is the release of hemoglobin from ruptured red blood cells (erythrocytes) into blood plasma. Hemolysis is a frequent complication associated with implantation of prostheses, which forms a significant barrier to healing due to risk of hemoglobinemia and can lead to increased risk of infection due to its inhibitory effects on innate immunity. In the standard method, release of hemoglobin is determined by measuring the total amount of hemoglobin present in a given blood sample and comparing this to the free hemoglobin in the plasma after the undamaged red cells are removed by centrifugation. The plasma remaining is reacted with Drabkin's solution to form cyanmethemoglobin.

In the analysis, rabbit blood was used to determine the total amount of hemoglobin present in the plasma and in the whole blood fraction. High density polyethylene (HDPE) was used as a negative control and nitrile rubber (Buna-N rubber) as a positive control to ensure the test reliability.

Test specimens were assigned a hemolytic index calculated by subtracting the corrected hemolysis % of the negative control from the corrected hemolysis % of the tested item. Hemolytic index range within 0-2% (above the negative control) is interpreted to indicate a non-hemolytic grade; 2-5%—a slightly hemolytic grade; and exceeding 5%—a hemolytic grade.

The results have demonstrated all coatings are hemocompatible. Sample SiO₂ was found to be slightly hemolytic, all the other samples and the non-coated reference were non-hemolytic.

The results presented above are clearly indicative of suitability of the laminate coating solutions according to the present disclosure in protecting medical devices intended for being continuously exposed to biological fluids, in particular, body fluid media. Tested coating compounds were biocompatible, non-toxic and additionally possessed antimicrobial activities.

The invention further pertains to an item, comprising a laminate coating according to the embodiments. The coating advantageously forms an ion diffusion barrier to prevent ionic species originating from essentially saline environments, optionally, in vivo environments, from penetrating through a plurality of deposition layers making the coating.

The item can be configured as a medical device, in particular, a body implantable medical device, optionally including functional electronic components to develop so-called (bio)electronics solutions. The item can thus be configured as an implant absent of electronic components (one-part or multipart implant) or a body implantable medical device that contains electrical/electronic systems, optionally self-powered systems. Implantable medical devices may be designed such as to completely reside in the body, or it may be provided external to the body and connect to an internal organ or another body part. Such devices include, but are not limited to implantable cardiac pacemakers, monitors and defibrillators (e.g. implantable cardioverter defibrillators, ICDs), cochlear implants, a variety of neurological implants and stimulators, infusion pumps, haemodynamic systems, micro-electrical mechanical systems (MEMS), and a variety of (miniaturized) sensors including biosensors for measuring pressure, flow, strain, etc., as well as (bio)chemical sensors, such as the ones for use for example in the treatment of diabetes (e.g. sensors for continuous glucose monitoring, CGM), and other chemically regulated conditions.

In terms of functionality, the implantable devices include a variety of monitoring and/or metering devices, such as the devices for measuring the levels of chemical substances, which serve as markers for certain diseases, in biological fluids, and the devices applicable in treatment of certain conditions or syndromes through neuromodulation (stimulators used in the treatment of migraine, for example) or through regulating the levels of said chemical substances in biological fluids.

The item can be also provided as a part or a component used in fabrication of optionally miniaturized sensors and/or semiconductor devices. The item can be further provided as a Printed Circuit Board (PCB) or a PCB assembly (PCBA). The item can be further provided as a non-invasive (medical) device or a part thereof (e.g. a wearable sensor (sensor system) or a semiconductor component/PCB(A)).

Still further, the invention pertains to a medical device, such as a body implantable medical device. The medical devices described hereinabove and similar to those, as well as any related items configured implantable into the body can be conceived. In embodiments, the medical device is rendered antimicrobial by virtue of chemical composition of a plurality of deposition layers forming the laminate coating deposited on the medical device, and having endotoxin release values less than or equal to 20 Endotoxin Units (EU) per a coated device, preferably, less than or equal to 2.15 EU per a coated device, as determined by the Limulus Amebocyte Lysate (LAL) assay.

It shall be appreciated by those skilled in the art that with the advancement of technology the basic ideas of the present invention may be implemented and combined in various ways. The invention and its embodiments are thus not limited to the examples described hereinabove, instead they may generally vary within the scope of the claims.

1. A laminate coating for substrates susceptible to corrosion in essentially saline environments, the coating comprising a plurality of deposition layers formed through a process of chemical deposition in vapour phase, such that the deposition layers having a first composition alternate with the deposition layers having a second composition different from the first composition, wherein said plurality of deposition layers form a diffusion barrier that prevents corrosive species originating from essentially saline environments from contacting the substrate.

2. The laminate coating of claim 1, in which the individual deposition layers at least partially block the diffusion of corrosive species therethrough.

3. The laminate coating of claim 1, comprising at least one ion-conductive deposition layer enabling selective diffusion of corrosive species originating from essentially saline environments therethrough.

4. The laminate coating of claim 1, further comprising at least one deposition layer reactive to corrosive species originating from essentially saline environments.

5. The laminate coating of claim 1, wherein the ion-conductive deposition layers alternate with the deposition layers reactive to diffusing corrosive species.

6. The laminate coating of claim 1, wherein interaction between said reactive deposition layer(s) and the corrosive species originating from the essentially saline environment yields formation of at least one additional barrier layer having composition different from that of any one of the deposition layers forming the laminate coating.

7. The laminate coating of claim 1, wherein the additional barrier layer is formed at an interface between the conductive deposition layer and the deposition layer reactive to diffusing corrosive species.

8. The laminate coating of claim 1, wherein the individual deposition layers are composed of any compound selected from the group consisting of: aluminium(III) oxide (Al₂O₃), titanium(IV) oxide (TiO₂), hafnium(IV) oxide (HfO₂), tantalum(V) oxide (Ta₂O₅), zirconium(IV) oxide (ZrO₂), and silicon dioxide (SiO₂).

9. The laminate coating of claim 1, comprising a deposition layer composed of aluminium(III) oxide (Al₂O₃) as a substrate adjacent layer.

10. The laminate coating of claim 1, comprising the deposition layers composed of aluminium(III) oxide (Al₂O₃) alternating with the deposition layers of zirconium(IV) oxide (ZrO₂).

11. The laminate coating of claim 1, comprising the deposition layers composed of hafnium(IV) oxide (HfO₂) alternating with the deposition layers of silicon dioxide (SiO₂).

12. The laminate coating of claim 1, wherein a stack formed with a plurality of deposition layers has thickness within a range of about 10 nm to about 300 nm.

13. A method for improving resistance of a substrate to corrosion in essentially saline environments, the method comprises:
(i) obtaining a substrate; and
(ii) forming, on said substrate, a laminate coating by depositing, through a process of chemical deposition in vapour phase, a plurality of deposition layers such, that the deposition layers having a first composition alternate with the deposition layers having a second composition different from the first composition, wherein said plurality of deposition layers forms a diffusion barrier that prevents corrosive species originating from essentially saline environments from contacting the substrate.

14. The method of claim 13, comprising depositing a plurality of deposition layers, which at least partially block the diffusion of corrosive species therethrough.

15. The method of claim 13, comprising forming the laminate coating with at least one conductive deposition layer that enables selective diffusion of corrosive species originating from essentially saline environments therethrough.

16. The method of claim 13, comprising forming the laminate coating with at least one deposition layer reactive to corrosive species originating from essentially saline environments.

17. The method of claim 13, wherein formation of the diffusion barrier involves chemical interactions between the deposition layer(s) and diffusing corrosive species.

18. The method of claim 13, further comprising (iii) exposing the substrate deposited with the laminate coating to an essentially saline environment.

19. The method of claim 18, wherein the essentially saline environment is an in vivo environment.

20. The method of claim 13, wherein an additional barrier layer is formed when the substrate deposited with said laminate coating is exposed to the essentially saline environment, the additional barrier layer having a composition different from that of any one of the deposition layers forming the laminate coating.

21. The method of claim 20, wherein the additional barrier layer is formed at an interface between the ion-conductive deposition layer and the deposition layer reactive to diffusing corrosive species.

22. The method of claim 13, wherein the individual deposition layers of the plurality of deposition layers formed on the substrate are composed of any compound selected from the group consisting of: aluminium(III) oxide (Al₂O₃), titanium(IV) oxide (TiO₂), hafnium(IV) oxide (HfO₂), tantalum(V) oxide (Ta₂O₅), zirconium(IV) oxide (ZrO₂), silicon dioxide (SiO₂), or any combination thereof.

23. The method of claim 13, comprising formation of a deposition layer composed of aluminium(III) oxide (Al₂O₃) on a substrate surface.

24. The method of claim 13, wherein the plurality of deposition layers formed on the substrate includes the deposition layers composed of aluminium(III) oxide (Al₂O₃) alternating with the deposition layers of zirconium(IV) oxide (ZrO₂).

25. The method of claim 13, wherein the plurality of deposition layers formed on the substrate includes the deposition layers composed of hafnium(IV) oxide (HfO₂) alternating with the deposition layers of silicon dioxide (SiO₂).

26. A barrier to diffusion of corrosive species towards the substrate in essentially saline environments, the barrier comprising the laminate coating of claim 1.

27. A method for extending the lifetime of a substrate exposed to essentially saline environments, the method comprising applying the laminate coating of claim 1 to the substrate.

28. An item, comprising a laminate coating as defined in claim 1, said coating forming a diffusion barrier to prevent corrosive species originating from essentially saline environments from penetrating through said laminate coating.

29. A medical device configured as the item of claim 28, said medical device rendered antimicrobial by virtue of chemical composition of a plurality of deposition layers forming the laminate coating, and having endotoxin release values less than or equal to 2.15 Endotoxin Units (EU) per a coated device as determined by the Limulus Amebocyte Lysate (LAL) assay.
